(19) United States
(12) Patent Application Publication
(10) Pub. No.: US 2008/0257348 A1
(43) Pub. Date: Oct. 23, 2008

(54) Emergency and mass casualty ventilator

(76) Inventor: S. David Piper, Carmichael, CA (US)

Correspondence Address: David Piper, P.O. Box 993, Carmichael, CA, 95609, US

(21) Appl. No.: 12/148,734

(22) Filed: Apr. 21, 2008

Related U.S. Application Data
(60) Provisional application No. 60/913,190, filed on Apr. 20, 2007.
(60) Provisional application No. 60/916,211, filed on May 4, 2007.

(51) Int. Cl. A61M 16/00; A62B 18/02

(52) U.S. Cl. 128/204.18; 128/204.21; 128/205.25

(57) ABSTRACT

This invention pertains generally to ventilation devices, and more particularly to a low cost constant pressure, variable flow blower powered ventilation device that is fully functional with a mask and which can be adapted to a number of applications including (1) a respiratory device used for automatic resuscitation of patients needing emergency ventilation, (2) emergency backup ventilation capabilities for hospitals and other healthcare institutions, and (3) positive pressure support therapy for patients suffering from obstructive sleep apnea among other diseases and respiratory conditions.

This application claims the benefit under 35 U.S.C. 119(e) of U.S. provisional application Ser. No. 60/913,190 filed Apr. 20, 2007, and No. 60/916,211 filed May 4, 2007, which are each incorporated by reference herein in its entirety.

### BACKGROUND OF THE INVENTION

A fundamental aspect of providing respiratory care to a patient is the ability to provide ventilatory support to patients requiring respiratory assistance. Ventilatory support can be defined as providing a periodic flow into and out of the patient in such a manner to allow the lungs to inflate and deflate, thus continuing the oxygenation of blood in proximity to the lungs. Ventilatory support may be required in a number of situations, including but not limited to; the scene of an emergency or accident, an ambulance, the home, the emergency room, the halls of a hospital during transport, in the Intensive Care Unit (ICU) of a hospital, or at the scene of a mass casualty event. Ventilatory support can be provided in a number of different ways, such as by a manual resuscitator, First-Aid Cardio Pulmonary Resuscitation (CPR), use of an automatic ventilatory device, or use of an automatic resuscitator. Decisions as to which device to use is dependent on equipment availability and the personnel resources obtainable to effectively operate the chosen device within necessary functional controls.

Manual resuscitators are typically equipped with a self-inflating bag, a set of check valves (or a duckbill valve) on the inlet circuit which control the direction of inhalation and exhalation gases, and a patient interface which is usually either a face mask or a port for connection to an endotracheal tube. An operator of a manual resuscitator introduces oxygen enriched therapeutic gas into the patient's lungs by applying a constrictive force to the self-inflating bag. As the operator terminates the constrictive force and the self-inflating bag is allowed to refill, pressure of the introduced gas combined with the elastic nature of the patient's own respiratory system causes the introduced gas to then be expelled through the patient's airway and past the check-valves in the manual resuscitator.

Most manual resuscitators are equipped with means to maintain a small minimum positive pressure in the patient's lungs and airways so as to maintain that airway in an “open” condition. This minimal positive pressure is commonly referred to as the “Positive End Expiratory Pressure” or “PEEP”. Upon conclusion of the exhalation phase wherein the patient's respiratory system returns to an ambient pressure, in conjunction with the additional PEEP, the operator again constricts the self-inflating bag, the check-valves on the inlet circuit re-open and the process is repeated.

The ubiquitous practice of manual type resuscitators is evident in the fact that little skill is required to effect cyclic respiration and by the relatively inexpensive nature of such an uncomplicated device. Unfortunately, manual resuscitators can be, and often are, misused and/or misapplied, as there is no means within the device for ensuring proper recycle time or appropriate duration of either the inhalation or the exhalation phases. A number of studies have been published which show that irrespective of the degree of operator training (as evident in whether the operator of the manual resuscitator is a physician, respiratory therapist, or nurse), patients generally receive volumes of gas per breath, referred to as a “tidal volume”, which are too small and/or are provided to the patient at respiratory rates which are too fast for effective respiration to occur. Inappropriate management of tidal volume has been shown to create significant adverse effects on patients. Representative published journal articles directed to such issues with misuse of manual type resuscitators include “Evaluation of 16 adult disposable manual resuscitators”, Mazzolini DG Jr et al., Respiratory Care. 2004 December; 49(12):1509-14 and “Miss-located pop-off valve can produce airway overpressure in manual resuscitator breathing circuits,” Health Devices, 1996 May-June; 25(5-6):212-4, both of which are incorporated by reference in their entireties. Furthermore, manual resuscitators have the additional disadvantage of requiring the continuous use of the rescuers hands, limiting their usefulness for a mass casualty event.

First-Aid Cardio Pulmonary Resuscitation (CPR) is a widely taught technique of resuscitation for patients needing respiratory assistance. However, it has the disadvantage of requiring the continuous use of the rescuers hands, the need for a second rescuer, and provides inadequate ventilatory support. Representative published journal articles directed to such issues with manually applied CPR include “Optimum cardiopulmonary resuscitation for basic and advanced life support: a simulation study”, Turner et al., Resuscitation. 2004 August; 62(2):209-17 and “Importance of continuous chest compressions during cardiopulmonary resuscitation: improved outcome during a simulated single lay-rescuer scenario”, Kern et al., Circulation, 2002 Feb. 5; 105(5):645-9, both of which are incorporated by reference in their entireties. In reference to the article by Kern et al. 2002, the researchers found that ventilation performed during CPR created interruptions in chest compression-supported circulation and that the minimization of these interruptions would greatly improve patient outcome. Within the medical arts, CPR is simply intended as a technique of last and desperate resort, and not as a substitute for a device capable of delivering sufficient ventilatory support. Further, due to the significant demand CPR requires on qualified personnel, the method is certainly not suitable for a mass casualty event whereby qualified personnel will be vastly outnumbered versus patient demand.

There are a large number of different types of automatic ventilatory devices (ventilators). The most prevalent type of ventilator uses sophisticated electronic logic circuits and is equipped with one or more flow and pressure sensors for monitoring the patient. These devices have the distinct advantage that they do not require the constant use of the clinician's hands and are usually equipped with numerous alarm features. BiPAP ventilators, often used for the treatment of sleep apnea, fall into this category of ventilatory support device. Electronic logic circuit driven ventilators can be used to deliver set volumes, flow, and/or pressure as intended by the device and set by the clinician. Patient pressure and/or flow is measured by the sensors, the signal of which is compared to the set point as determined or acquired by the logic circuit algorithm and the resulting difference is processed by electronic components the result of which is a electronic signal sent to increase or decrease the flow of breathable gas to the patient as controlled by a valve, the speed of a pressure generator motor, or similar such device or combination thereof.

Examples of the aforementioned logic controlled ventilators are common in the prior art. U.S. Pat. No. 6,895,962 to Kullik et al., utilizes a control unit to control a compressor relative to a pressure curve so as to obtain a desired flow rate through a filter unit. U.S. Pat. No. 5,211,170 to Press, teaches a ventilator unit for replacing the mouth-to-mouth aspect of CPR through use of a compressor controlled by a pressure sensor. Published U.S. Patent Application No. 2006196508 to Chalvignac teaches to a variable rate motor for controlling air pressure at a given flow.

Although these devices are capable of delivering adequate respiratory support in a manner that does not require the constant attention of the clinician, they are very expensive because of their expensive sensors and pressure/flow control devices and require extensive clinical training before they can be used in a safe and effective manner. Consequently, they are largely unsuitable as a device for emergency first response, due to the sophistication of the training required for use, and are unsuitable for a mass casualty event given their large capital costs.

Automatic resuscitator is a class of ventilator/device that is intended to meet the needs of emergency respiratory support. Automatic resuscitators are generally simpler than most ventilators. Unfortunately, the capital costs for most of these devices are prohibitive to community preparation, let alone sudden or massive casualty event. Furthermore, these devices often require sophisticated clinicians to be operated effectively, making them further unavailable for a wide array of first responders to an emergency situation. Many of these devices are also gas powered, making them unsuitable for a mass casualty event due to the overwhelming logistics of providing enough necessary compressed medical gas.

Further compounding the issue with automatic resuscitators is the tendency of such devices to experience deleterious leakage at the mask interface with the patient's face. Such leakage is particularly problematic in that control logic will either render the resuscitator non-operational through an error condition or will be in an inefficient, if not otherwise dangerous to patient outcome, constant on or limited function status. Automatic resuscitators that are simpler in design, and thus enjoy a smaller capital cost (such as the VAR manufactured by Vortran Medical Technology, U.S. Pat. No. 6,067,984) also do not have the ability to compensate for leaks between the patient and the patients mask, and thus require the constant application of pressure by the user onto mask and the patient, and are therefore not a hands free device. Prior art attempts, such as U.S. Pat. No. 5,510,222 to Younes, to address leakage involve use of sensors and logic paths to evaluate the degree of leak and compensate for the leak by altering the performance of the resuscitator. The necessary complexity inherent to such evaluation and compensation for leakage, again, complicates the design of the resuscitator and thereby increases the cost of procuring such a device.

The medical infrastructure of the United States, and much of the rest of the world, is based on the presumption that existing highly trained clinicians will outnumber and readily have access to almost all patients requiring emergency assistance, and that the capital equipment costs to provide emergency care to these patients will be relatively small due to the small number of patients needing care at any one time. Historically, emergency medical response has been limited to cases of car accidents, heart attacks, and other events that are individual in nature and which the existing medical infrastructure has been well suited. Recently, new threats such as terrorism, natural disasters, and pending flu epidemics have been identified as likely future events that are projected to produce large numbers of patients in a short period of time (a mass casualty event). During a mass casualty event, patients will significantly outnumber existing trained clinicians, existing trained clinicians may have little or no access to patients, and existing capital equipment will be insufficient.

Therefore, a need exists for a ventilator support device that has a minimal degree of complexity and corresponding reliance on sensor based operational formatting, requires limited training and hands-on use to effectively utilize, requires no compressed gas supplies and is readily powered by electricity, and can compensate for patient mask leaks without changing base function.

### BRIEF SUMMARY OF THE INVENTION

This invention pertains generally to ventilation devices, and more particularly to a constant pressure, variable flow, electrically powered centrifugal compressor ventilation device that is fully functional with a mask and which can be adapted to a number of applications including (1) a respiratory device used for automatic resuscitation of patients needing emergency ventilation, (2) emergency backup ventilation capabilities for hospitals and other healthcare institutions, and (3) positive pressure support therapy for patients suffering from obstructive respiratory conditions such as sleep apnea among other diseases.

The central component of the present invention employs a centrifugal compressor and specifically a turbine blower with a turbine element having specific functional attributes such that it provides a constant pressure over a wide range of flow rates. In the preferred embodiment, the turbine blower will provide a useful operation pressure range from a minimum pressure of 0 cm-water to a maximum pressure of 33 cm-water, and with a flow rate from zero to 100 liter per minute. Based on the setting of the turbine blower to a specific pressure within the operational pressure range, the set pressure point will exhibit a tolerance of +/−5% across the entirety of the flow range. This pressure versus flow rate performance offers the distinct advantage of being able to provide a known and set peak pressure to a patient, provide for automatic leak compensation between the patient and the patient mask without altering the turbine blower operation, and provide constant pressure ventilatory support for changing lung compliance conditions without any pressure sensors, flow sensors, servo controlled motors, pressure control valves, or other such sensor and control components that significantly add to the cost of other ventilation devices available on the market or previously described in prior art.

Variance in setting of the specific pressure for higher or lower pressure ventilation is possible by changes in the rotational speed and/or scale of the turbine element. Changes in the pressure have been shown to be proportional to the pressure parametric (PP) which is equal to the square of the product of the rotational speed (RS) and diameter of the turbine element (D). Similarly, the device can be made more or less able to compensate for leak through variance of the rotational speed and/or diameter of the turbine. Changes in the turbines ability to provide leak compensation were found to be proportional to the leak compensation parametric (LCP) which is equal to product of the rotational speed (RS) and the cube of the diameter of the turbine element (D).

In one of many possible embodiments of the current invention, the ventilation device of the present invention is supplied with electrical power, turned on by means of a simple power switch (e.g. rocker-type), and automatically provides by activation of the turbine blower a predetermined 20 cm water of peak pressure ventilatory support (optionally manually adjustable from 2 to 30 cm water) at sixteen (16) breaths per minute (optionally manually adjustable from 4 to 40 breaths per minute) as determined by the inspiratory time while the turbine blower is on and expiratory time while the turbine blower is off. The turbine blower is ducted via a standard 22 mm corrugated tube attached to a patient cushion-type mask that is attached to the patient's head via elastic straps attached to the mask and circumscribing the patient's head. Respiratory rate, as determined by inspiratory time plus expiratory time/60 seconds per minute, is controlled by a simple closed-circuit timing control that turns the motor powering the turbine element within the turbine blower on and off. As is well known, a control knob connected to a potentiometer can be used to allow the user to vary any of the control variables, if desired.

In one embodiment, the turbine blower is controlled by a simple direct current (DC) electrical motor equipped with an internal rectifier for use of alternating current (AC) electricity. Use of this DC/AC motor has the desired advantage of being substantially less expensive than other servo controlled motors and the like used in devices available on the market and described in the prior art. Optionally, a manometer, disposable or reusable in nature, can be used to monitor the operation of the device and to insure proper functioning. The device may be equipped with a manometer that contains a simple red (i.e. too low/too high) and green (i.e. within target) background signifying proper pressure operation or not.

In another embodiment, the present invention can be configured to allow for variation of patient peak pressure.

In another embodiment, the present invention can be configured to provide a maximum peak pressure and a Positive End Expiratory Pressure (PEEP).

In another embodiment, the present invention can be configured with control dials for controlling peak pressure, PEEP, respiratory rate.

In another embodiment, the present invention can be configured with control dials for controlling peak pressure, PEEP, respiratory rate, and the ratio of inspiratory time to expiratory time.

An object of the invention is to provide an emergency ventilation device that be used effectively during a mass casualty event.

Another object of the invention is to provide an emergency ventilation device that can be used easily by persons without significant training.

Another object of the invention is to provide an emergency ventilation device that is able to deliver the same performance in the case of a range of mask leaks between the patient and the patient mask.

Other features and advantages of the present invention will become readily apparent from the following detailed description, the accompanying drawings, and the appended claims.

### BRIEF DESCRIPTION OF THE SEVERAL VIEWS OF THE DRAWING(S)

The invention will be more easily understood by a detailed explanation of the invention including drawings. Accordingly, drawings, which are particularly suited for explaining the inventions, are attached herewith; however, it should be understood that such drawings are for descriptive purposes only and as thus are not necessarily to scale beyond the measurements provided. The drawings are briefly described as follows:

FIG. 1 is an exploded diagram of a ventilation device in accordance with the present invention.

FIG. 2 is a perspective view of a ventilation device.

FIG. 3 is a front view of a ventilation device.

FIG. 4 is a back view of a ventilation device.

FIG. 5 is a left side view of a ventilation device.

FIG. 6 is a right side view of a ventilation device.

FIG. 7 is a top down view of a ventilation device.

FIG. 8 is a bottom up view of a ventilation device.

FIG. 9 is a cross sectional side view of the ventilation device taken at the midline between the left and right sides.

FIG. 10 is perspective view of the blower unit subassembly.

FIG. 11 is a front view of the blower unit subassembly.

FIG. 12 is a bottom up view of the blower unit subassembly.

FIG. 13 is a right side view of the blower unit subassembly.

FIG. 14 is a perspective view of the lower turbine housing.

FIG. 15 is a bottom up view of the lower turbine housing with turbine structural fins.

FIG. 16 is a perspective view of the turbine element depicting the turbine blade height (BH), turbine blade outer twist angle (OTA) and turbine blade inner twist angle (ITA).

FIG. 17 is a down view of the turbine element depicting the turbine blade radius (BR), turbine blade outer angle (OA) and turbine blade inner angle (IA).

FIG. 18 is a graphic showing the angular speed of the of the centrifugal compressor in a representative inventive device relative to achieving higher flow rates.

FIG. 19 is a graphic showing the electrical current of the centrifugal compressor in a representative inventive device relative to achieving higher flow rates.

FIG. 20 is a graphic showing the pressure of the centrifugal compressor in a representative inventive device relative to achieving higher flow rates.

FIG. 21 is a graphic showing the angular speed of the of the centrifugal compressor in a representative inventive device relative to achieving higher flow rates.

FIG. 22 is a graphic showing the electrical current of the centrifugal compressor in a representative inventive device relative to achieving higher flow rates.

FIG. 23 is a graphic showing the pressure of the centrifugal compressor in a representative inventive device relative to achieving higher flow rates.

### DETAILED DESCRIPTION OF THE INVENTION

While the present invention is susceptible of embodiment in various forms, there is shown in the drawings and will hereinafter be described a presently preferred embodiment of the invention, with the understanding that the present disclosure is to be considered as an exemplification of the invention, and is not intended to limit the invention to the specific embodiment illustrated.

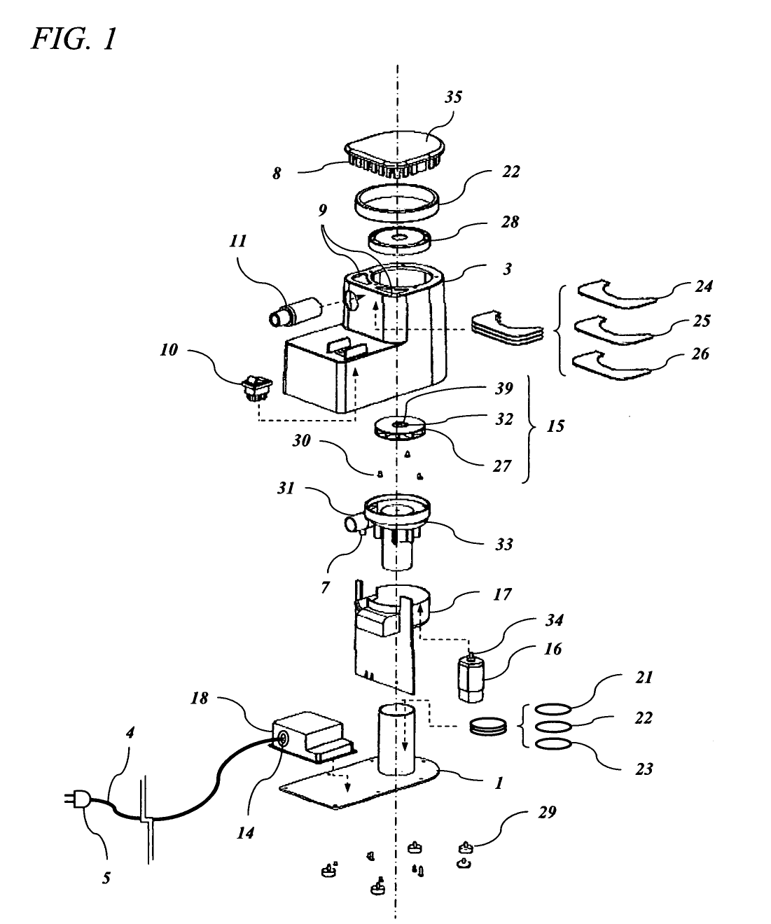

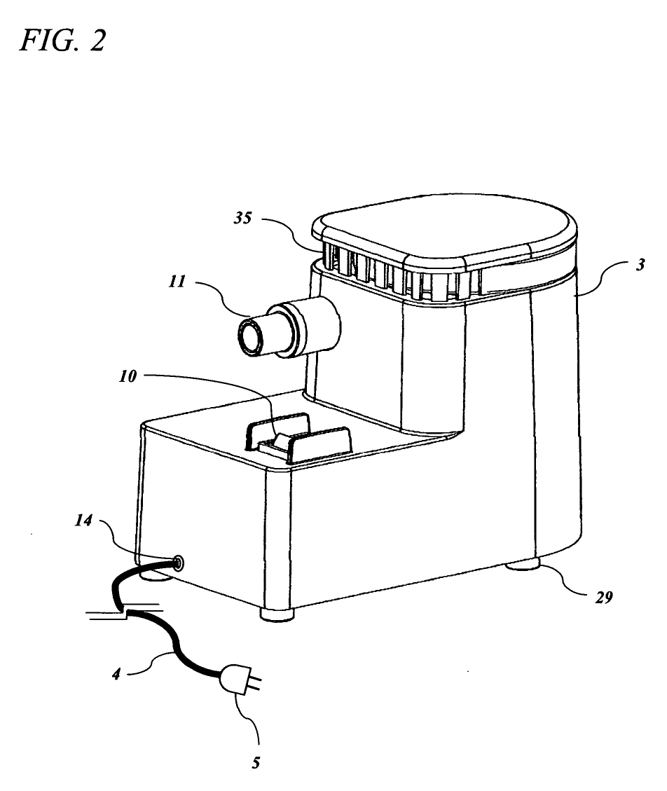

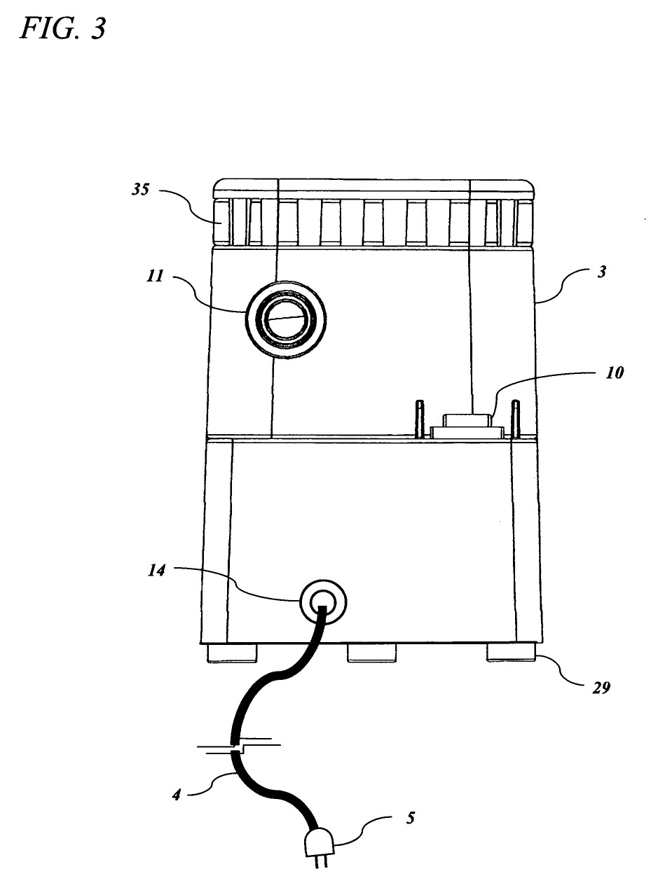

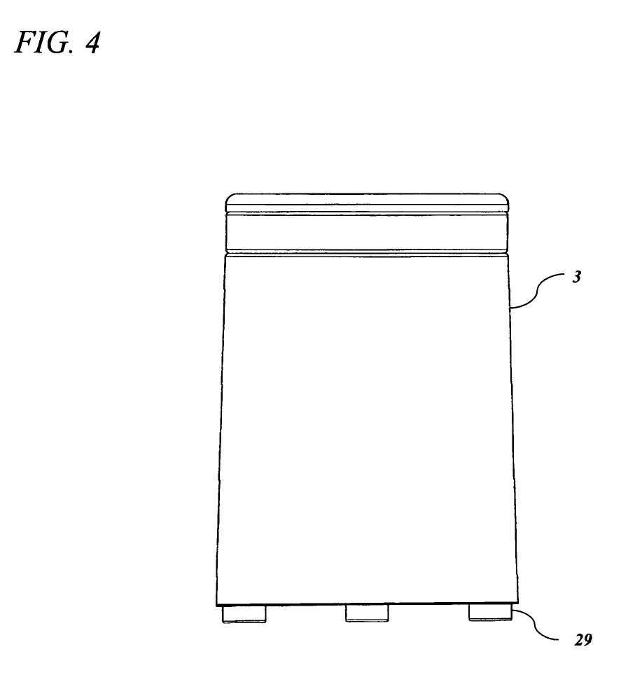

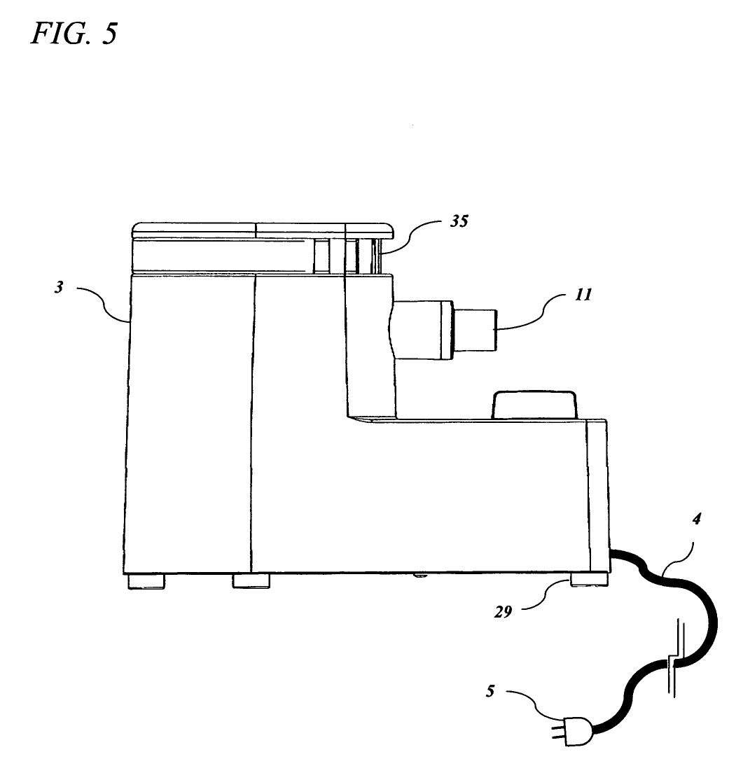

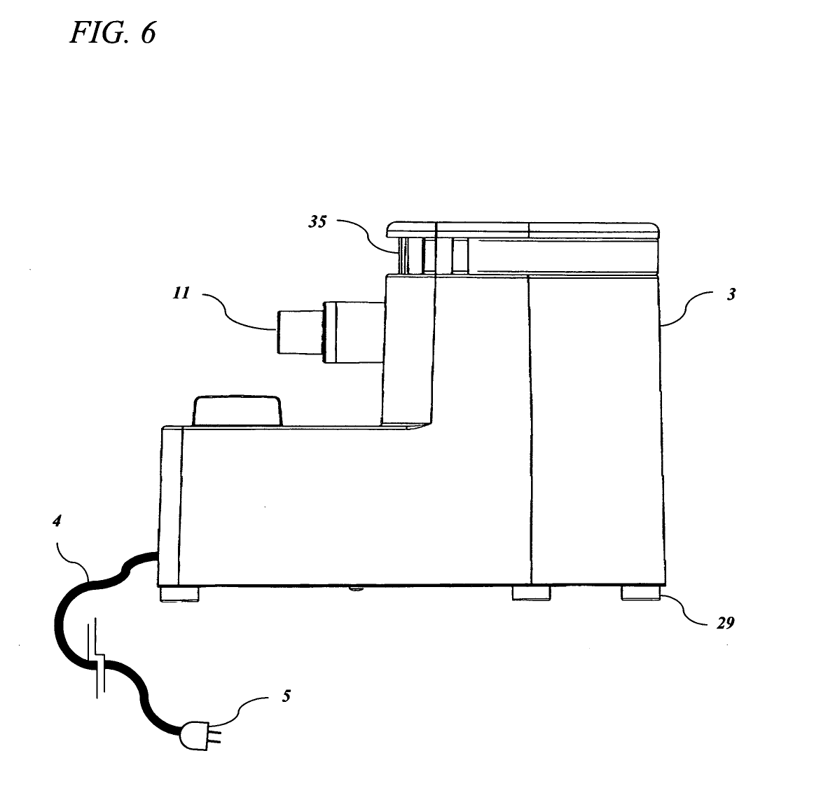

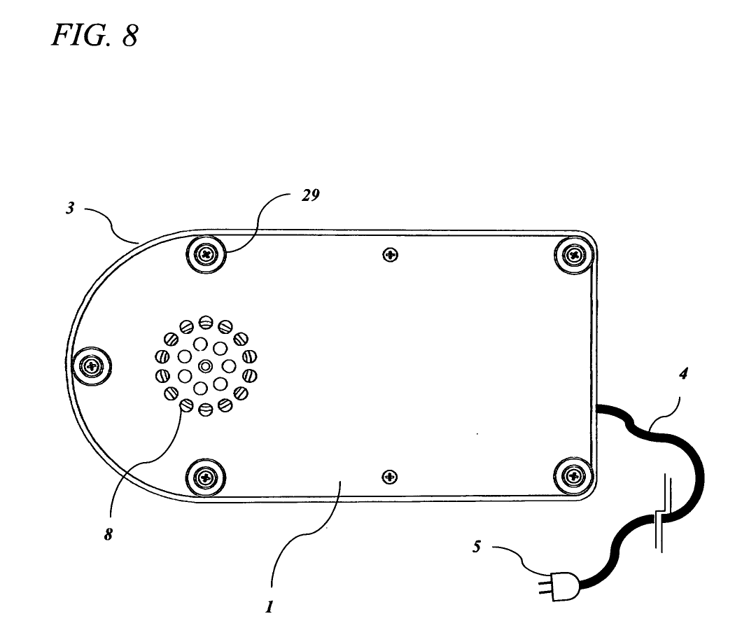

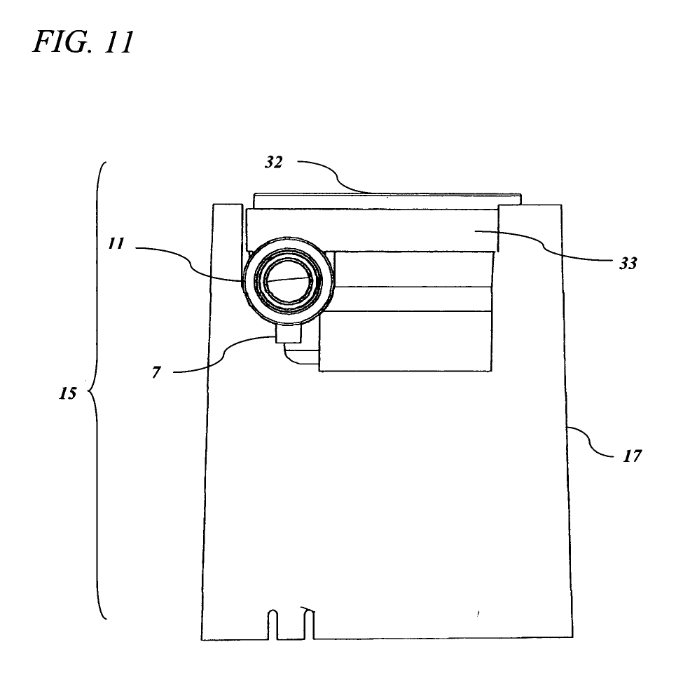

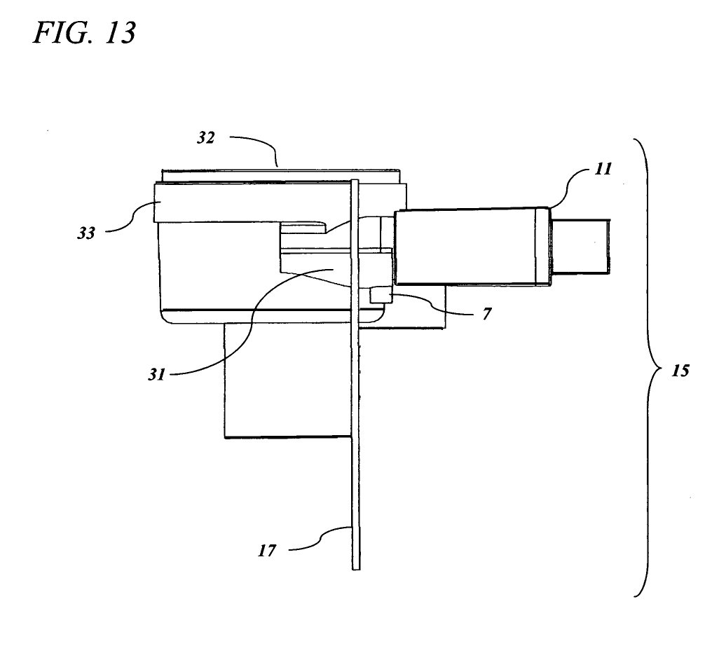

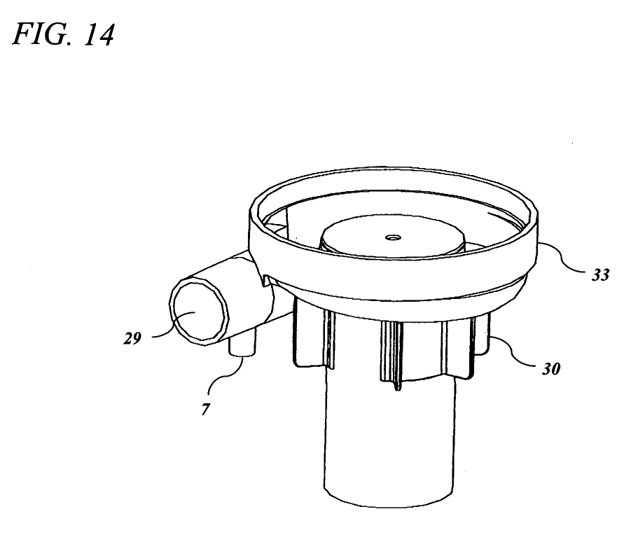

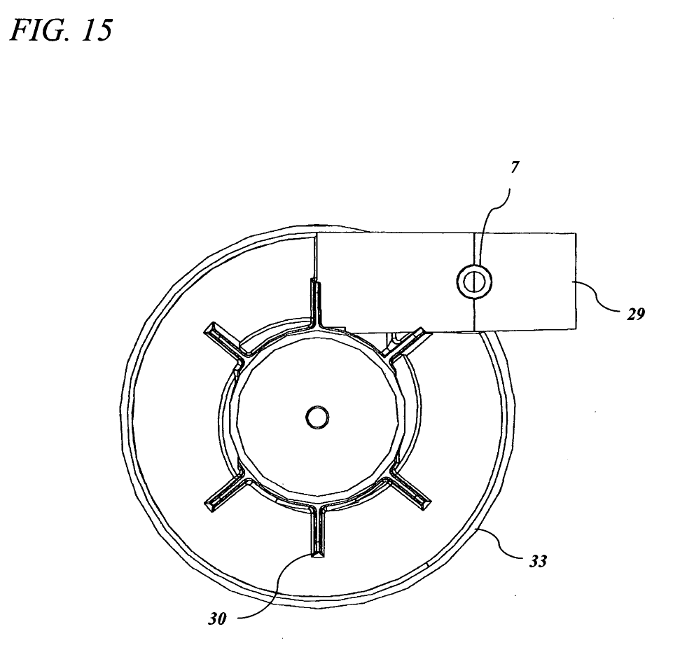

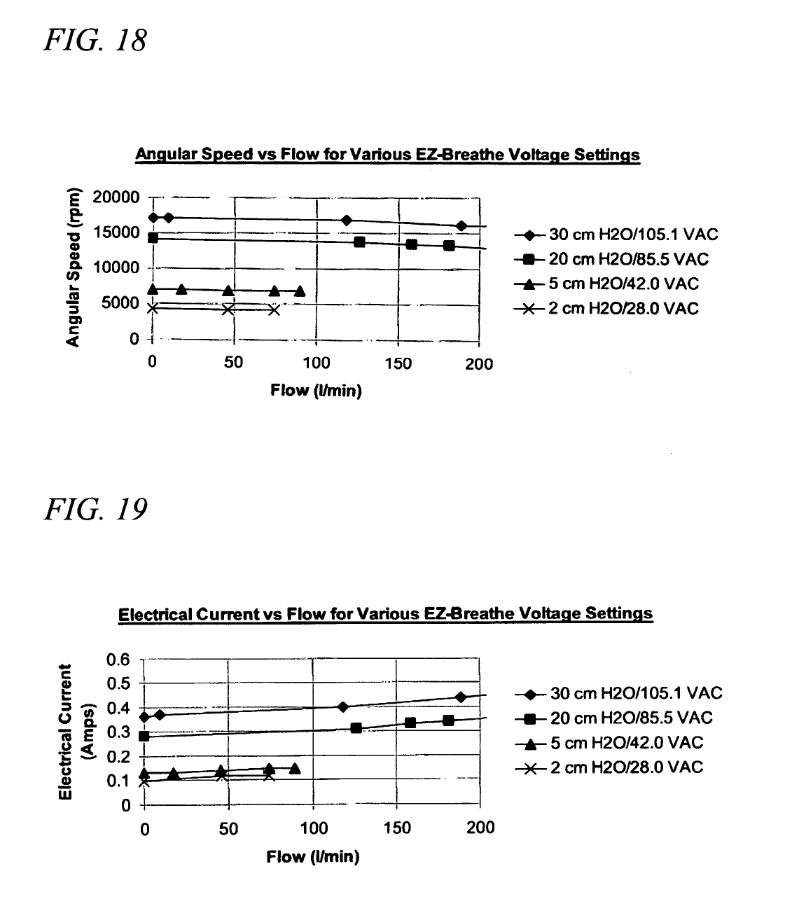

Referring more specifically to the figures, for illustrative purposes the present invention is embodied in the apparatus generally shown in FIG. 1 through FIG. 17 and graphs provided in FIG. 18 through FIG. 23.

As shown in FIG. 1, the device consists of a cabinet floor 1 (having attached thereto slip and vibration resistant feet 29), cabinet case 3, and ventilated cabinet cap 26. Electricity is provided through electrical cord 4 which is equipped with the appropriate plug 5 for access to 115 volts AC or similar such power source. The electricity is provided to an internal closed electric circuit through means of toggle switch 10. In the embodiment shown in FIG. 1, the invention has at least two predefined ventilator settings, which are controlled through toggle switch 10. Air is entrained by an internal turbine assembly 15 through air inlet 8, a small percent of which is fed through cooling port 7 to motor 16 for cooling and allowed to pass out the cooling vent 9. It will be appreciated that the apparatus may vary as to configuration and as to details of the parts without departing from the basic concepts as disclosed herein.

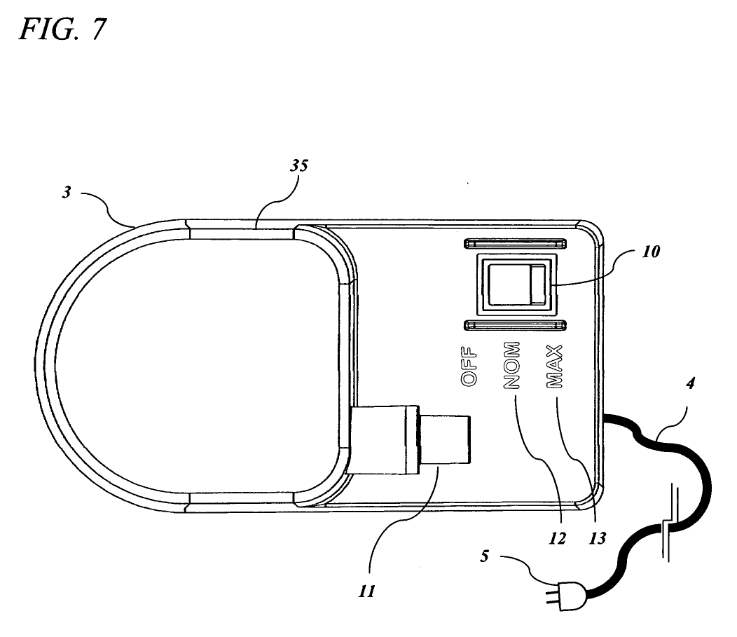

Toggle switch 10 is shown along with max ventilation label 12 and nominal ventilation label 13 (more clearly seen in FIG. 7), which both serve to indicate the necessary position of the toggle switch required for the described setting. Toggle switch 10 is further equipped with an internal LED that lights up when the device has been provided with power and the switch is in the “ON” position. Electrical cord 4 is equipped with a strain relief 14 at the base of power cord 4 where it interfaces with cabinet case 3. A fluid conduction pathway is established with the patient at the upper outlet extending through the cabinet case 3 at patient gas conduit connector 11. Nominally, the gas conduit connecter is capable of receiving fluid conduction pathways such as a medical grade or oxygen rated tubing having an internal diameter of 22 mm.

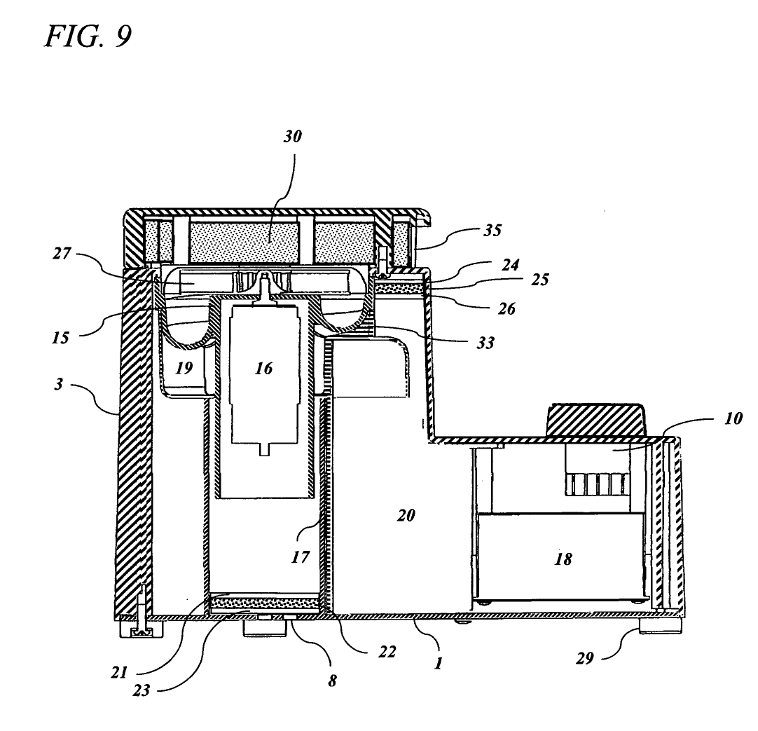

As shown in FIG. 1 and FIG. 9, turbine assembly 15 is shown along with motor 16, which are mounted to cabinet base 1 by way of bulkhead 17. Turbine assembly 15 and motor 16 are mounted on opposite sides of bulkhead 17. Bulkhead 17 separates control box 1 into two separate compartments: turbine compartment 19 and motor compartment 20 (FIG. 9). Electronic circuit 18 is located in motor compartment 20. Bulkhead 17 prevents any fluid communication between turbine compartment 19 and motor compartment 20 to prevent ozone produced by motor 16 from being entrained into turbine assembly 15 and subsequently supplied to the patient through patient gas conduit connector 11. Turbine assembly 15 is equipped with a small flow conduit, cooling port 7, to allow a small fraction of the airflow produced by the turbine to pass through the internal components of motor 16 thus providing essential cooling to motor 16, and purging motor compartment 20 of heat and ozone. Gas passing through motor 16 is allowed to exit cabinet case 3 through cooling vent 9. Patient gas conduit connector 11 serves as the gas outlet of turbine assembly 15.

In the exploded view, as shown in FIG. 1, turbine assembly 15 contains within the housing a turbine blade 27 that is mounted on central shaft 34 of motor 16 with no significant contact with turbine housing 33, allowing for relatively free motion of turbine blade 27 when driven by motor 16. The turbine housing 33 consisting of an outlet side 31 and cooling port 7. Retaining screws 30 maintain inlet side 32 in close proximity to turbine blade 27.

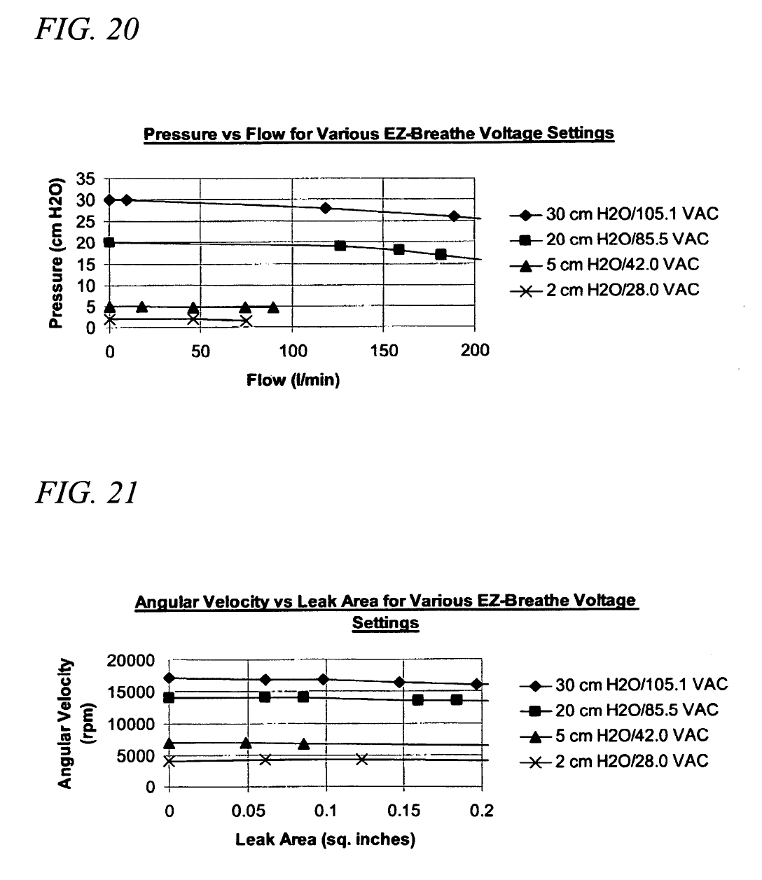

Motor 16 is a high speed, high voltage DC motor equipped with an internal rectifier for converting the AC electrical power provided by power cord 4 to the DC electrical power provided to the armatures of motor 16. In reference to FIG. 18, preferred motor types include those having an angular speed range of between 2,000 and 30,000 rotations per minute, preferably 3,000 and 20,000 rotations per minute, and most preferably 3,500 and 18,000 rotations per minute when operating a turbine assembly 15 with three-inch turbine blade 27 in accordance with the present invention. The angular speeds are based on the motor/turbine assembly attaining a target stagnation pressure (max pressure, zero flow) of 2, 5, 20, and 30 cm water. FIG. 19 presents the amperage draw of the same assemblies as used in FIG. 18. FIG. 20 depicts the unique nature of the present invention to provide a constant pressure (within 5% of target) at flow rates of up to 100 liters per minute flow. Further, it is preferred that motors capable of reaching target angular speed in one second or less are employed for enhanced responsiveness to being turned on and off by electrical circuit 18.

Returning to FIGS. 1 through 9, the ventilation device of the present invention is supplied with electrical power, turned on by means of a simple power switch (e.g. rocker-type), and automatically provides by activation of the turbine assembly 15 a predetermined 20 cm water of peak pressure ventilatory support (optionally predetermined or manually adjustable from 2 to 30 cm water). The ventilation device has the capability to cycle ON and OFF, corresponding to inspiratory and expiratory events, at sixteen (16) cycles or “breaths” per minute (optionally predetermined or manually adjustable from 4 to 40 breaths per minute) as defined by the inspiratory time while the turbine blower is running at a set voltage and expiratory time while the turbine blower is off. Electrical circuit 18 controls the inspiratory and expiratory time. Electrical circuit 18 is configured with a time controller embedded in a printed circuit board will a simple binary control path. The control path is provided by a relay or other simple oscillating time keeping mechanism such that the electrical circuit to motor 16 is trigger on and trigger off at predetermined rate by supplying a set voltage or no voltage. For example, the time oscillator may be used to set the exhalation time from a range of settings (i.e. from approximately 0.5 second to over 6 seconds). The timer may be set at the time of device manufacture, in the alternative, be controlled by a potentiometer and optional timer selection knob (not shown). The later embodiment allows an operator to set the desired exhalation time (and thereby the device controlled respiratory rate) or an inhalation to exhalation ratio. Additional potentiometers or other manually operated control devices known in the art may be included into the control path to allow for setting of peak pressure at a set voltage, residual pressure at a reduced voltage, and other such respiratory variables at the time of device manufacture or with suitable user interfaces as may be desired.

Referring to FIG. 9, motor 16 and turbine assembly 15 are shown as mounted in bulkhead 17, preventing fluid communication between motor compartment 20 and turbine compartment 19 except for the ancillary flow bled from turbine assembly 15 through cooling port 7 that is then fed through inside of the motor compartment 20 and then out cooling vent 9. Electronic circuit 18 is shown mounted on floor of cabinet base 1.

Continuing reference to FIG. 9, air inlet 8 consists of inlet lower screen 21, inlet air filter 22, and inlet upper screen 23. Inlet lower screen 21 and inlet upper screen 23 are perforated metal, or some such other material that is able to provide structural support to inlet air filter 22 which is an open cell foam material (usually polyurethane). Inlet lower screen 21, inlet air filter 22, and inlet upper screen 23 are affixed to control box 1 by use of screws and aligned with openings in the box to allow the passage of ambient air entrained into the box 1. Similarly, cooling vent 9 is also equipped with vent lower screen 24, vent filter 25, and vent upper screen 26.

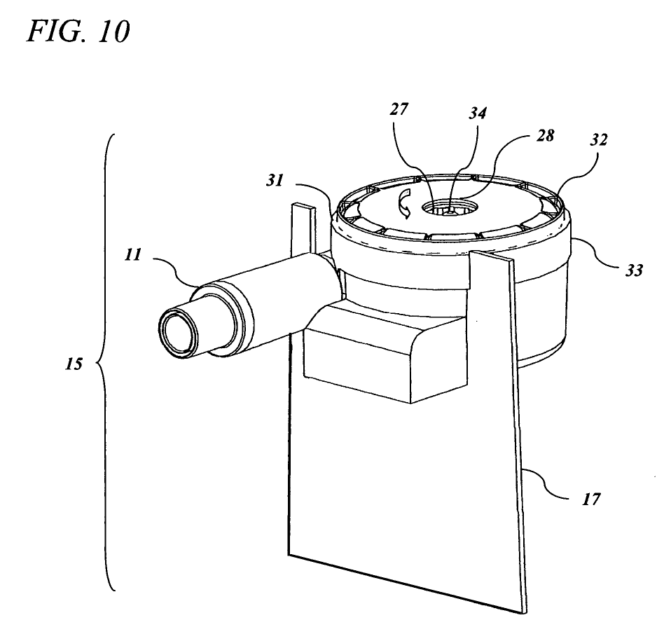

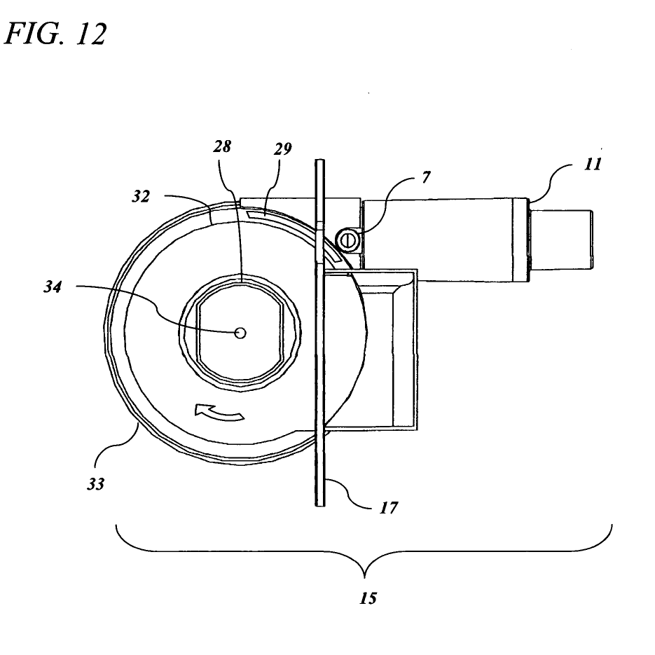

Referring to FIGS. 10 through 13 therein is depicted turbine assembly 15. FIG. 10 is a perspective view of the turbine assembly 15 with motor 16. The turbine assembly 15 contains a turbine housing 33 consisting of an outlet side 31 and an inlet side 32. In the assembled view, as shown, turbine assembly 15 contains within the house a turbine blade 27 that is mounted on central shaft 34 of motor 16 with no significant contact with the turbine housing 33, allowing for relatively free motion of turbine blade 27 when driven by motor 16. When motor 16 drives turbine blade 27, turbine blade 27 is caused to turn in a counter-clockwise direction when viewed from direction as shown. The centrifugal force imparted on the air or gas inside the turbine blade 27 causes the flow of air from turbine inlet 28 through turbine blades 27, and out turbine outlet 29 (FIG. 12). One or more turbine structural fins 30 may be provided to reinforce outlet side 31 of turbine assembly and provide necessary rigidity to mount motor 16.

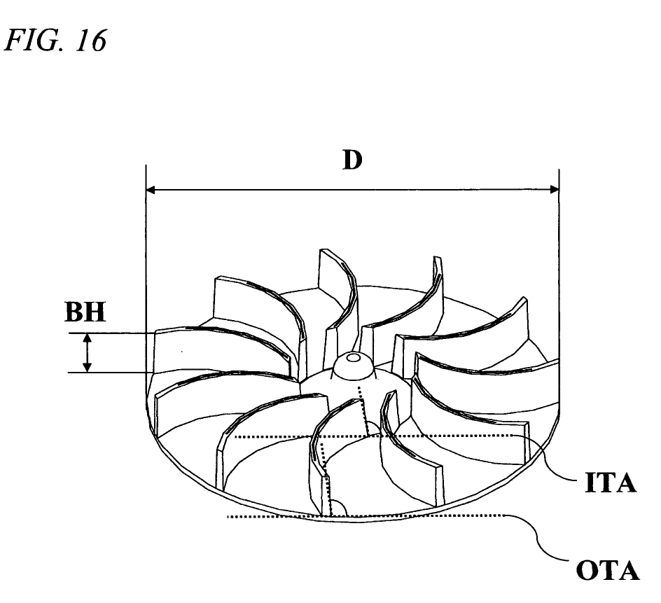

Referring to FIG. 16, turbine assembly 15 has been removed revealing a perspective view of turbine blade 27. Turbine blade 27 consists of planar turbine disk 40 having individual blade elements 41 extending from a central axis air turbine inlet 39 to an outer periphery of the planar turbine disk 40. At a turbine blade air inlet 36 position within air turbine inlet diameter 39. Centered within turbine blade air inlet 36 is turbine inlet nose 37. Turbine inlet nose 37 is sized and radiused at the base so as to change the direction of air entering the turbine blade inlet 36 axially out radially towards turbine blade control surfaces 41, turbine blade and outlet 38. The size of turbine inlet nose 37 is chosen so as to be suitably durable to repeated application of torque by motor 16, the mass of turbine blade 27 and the resistance caused by the entraining air. For representative purposes, the diameter of turbine inlet nose 37 may be about 0.9 inches on a 3.0-inch turbine blade 27 or approximately one-third of the turbine blade diameter (D). The cross-sectional specific geometry of turbine inlet nose 37 is not a constraint so long as the pressure and leak compensation parametric of the overall unit is achieved. In a preferred embodiment using a 3.0-inch turbine blade 27, turbine inlet nose 37 has a cross-sectional height of 0.3 inches and a radiused profile from a central point at the tip to the base

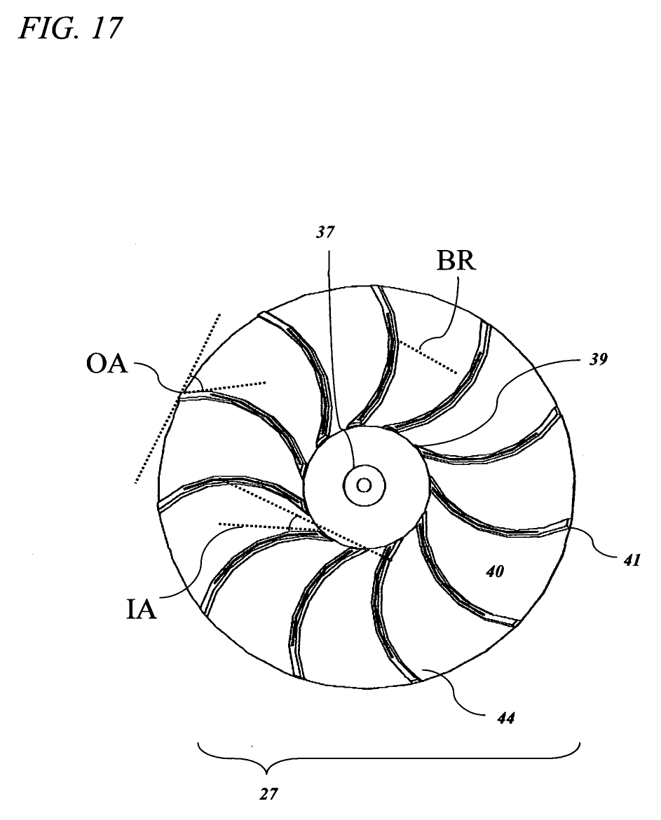

FIGS. 16 and 17 are perspective and top down views, respectively, of turbine blade 27 showing the critical turbine geometry of the invention. Table 1 identifies the upper and lower bounds from nominal for attributes of turbine blade 27 in attaining constant pressure with variable flow using a time-only controlled device. Turbine blade 27 is comprised of a plurality of blade control surfaces 41. Each blade control surface 41 has a height (i.e. blade height or BH) defined by the lower turbine blade surface 40 and extending upwards a finite amount. Each blade control surface 41 may be straight or exhibit simple or compound radii as defined by the blade radius or BR. The blade control surface 41 may exhibit a leading angle at the air turbine inlet 39 (defined as the inner angle or IA) and which may terminate on the outer periphery with an trailing angle (defined as the outer angle or OA). It has been found by the inventors that a inner angle that is less than the outer angle is particularly beneficial in achieving high air velocities from turbine blade 27. The blade control surface 41 may also exhibit changes in angle from the beginning point within the air turbine inlet 39 (inner twist angle, or ITA) wherein such twist may continue or change as the blade sweeps to the outer periphery of turbine blade 27 (as reflected by the outer twist angle or OTA). It should be understood that these values are representative of what may be used so as to meet the target pressure and leak compensation parametrics.

TABLE 1

| | Minimum | Nominal | Maximum |
|---|---|---|---|
| Turbine Geometry Variable | (unit) | (unit) | (unit) |
| Blade Height (BH) | 0.20 inch | 0.35 inch | 0.75 inch |
| Blade Radius (BR) | 0.50 inch | 0.70 inch | ∞ (i.e. straight) |
| Turbine Diameter (D) | 2.5 inch | 3.0 inch | 5.5 inch |
| Blade Outer Angle (OA) | 10° | 60° | 90° |
| Blade Inner Angle (IA) | 10° | 25° | 90° |
| Blade Outer Twist Angle (OTA) | 35° | 90° | 145° |
| Blade Inner Twist Angle (ITA) | 35° | 90° | 145° |

Tables 2 and 3 identify nominal values for the pressure parametric (PP) and leak compensation parametric (LCP) based on rotational speed and diameter respectively.

The pressure parametric (PP) is a unit-less measure as defined by the following equation:

PP=(RS radian/sec×D inches) superscript (2)

wherein RS is the rotational speed of the turbine element and D is the diameter of the turbine element.

The leak compensation parametric (LCP) is a unit-less measure as defined by the following equation:

LCP=RS radian/second×D inches (superscript)3

TABLE 2

| Rotation Speed of | | |
|---|---|---|
| Turbine Element | | |
| (rpm) | | |
| D = 3.0 inches | PP | LCP |
| 2,000 | 394,635 | 5,654 |
| 5,000 | 2,466,470 | 14,135 |
| 8,000 | 6,314,164 | 22,615 |
| 10,000 | 9,865,881 | 28,269 |
| 12,500 | 16,673,339 | 36,750 |
| 15,000 | 22,198,232 | 42,404 |
| 17,500 | 31,965,454 | 50,884 |
| 20,000 | 39,463,524 | 56,538 |
| 22,500 | 52,190,510 | 65,019 |
| 25,000 | 61,661,756 | 70,673 |
| 27,500 | 71,922,272 | 76,326 |
| 30,000 | 88,792,929 | 84,807 |

TABLE 3

| Diameter of Turbine Element | | |
|---|---|---|
| (inches) | | |
| RS = 18,000 rpm | PP | LCP |
| 1.5 | 7,991,364 | 6,361 |
| 1.8 | 11,507,564 | 10,991 |
| 2.0 | 14,206,869 | 15,077 |
| 2.3 | 18,788,584 | 22,930 |
| 2.5 | 22,198,232 | 29,447 |
| 2.8 | 27,845,463 | 41,371 |
| 3.0 | 31,965,454 | 50,884 |
| 3.3 | 38,678,200 | 67,727 |
| 3.5 | 43,508,535 | 80,802 |

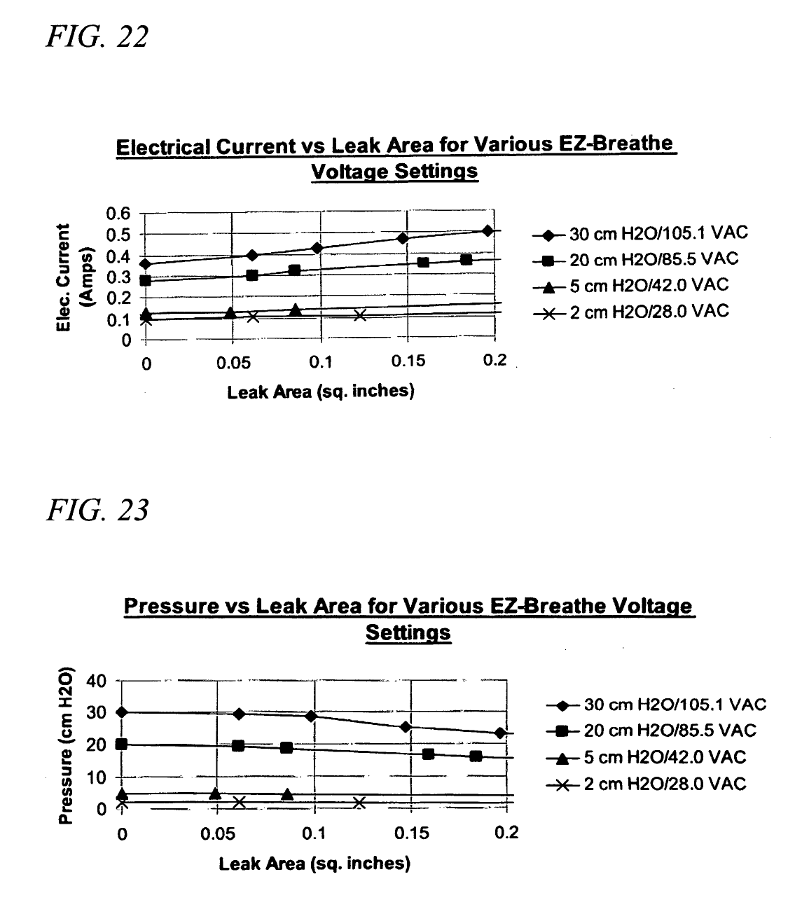

FIGS. 21 through 23 identifies the nominal for a given pressure with degree of flow capability and leak compensation using a time-only controlled device. In reference to FIG. 21, therein is shown angular velocity of the turbine blade 27 having a three-inch diameter and in accordance with preferred embodiment described herein affixed to motor 16 is essentially unaffected by incorporation of a leak of up to 0.2 square inch into the system. The leak was created by installing a ball-type valve into the patient conduit and intruding a leak of known area. The angular speeds are based on the motor/turbine assembly attaining a target stagnation pressure (max pressure, zero flow) of 2, 5, 20, and 30 cm water. FIG. 22 presents the amperage draw of motor 16/turbine blade 27 assembly at the same test points as listed in FIG. 21. As shown in FIG. 23, the motor 17/turbine blade 27 assembly of FIG. 21 shows there to be less than or equal to a 10 percent drop in delivered air pressure where the leak is less than or equal to 0.1 square inches, and less than or equal to a 20 percent drop in delivered air pressure where the leak is less than or equal to 0.2 square inches. In summary, through comparison of FIGS. 18 through 23, the inventive device disclosed herein has the capability of supplying an essentially constant pressure at flow rates of up to 100 liters per minute, and such device is able to maintain such performance within 10 percent of target pressure despite leakage within the system of up to 0.1 square inches.

The general construction of functional elements of the centrifugal compressor ventilator, as well as casing and control surfaces, may comprise polymer, nonferrous or ferrous compositions. Preferably, the functional elements are fabricated from suitable medical service, oxygen rated materials such as K-resin and ABS plastics.

### EXAMPLE

A first device was manufactured in accordance with teachings of the present invention. A first embodiment included a turbine inlet diameter 39 measures approximately 0.920-inch. The overall radius of turbine blade 27 is 1.4 inches making the diameter of turbine blade surface 40 equal to 2.8 inches. Turbine control blade surfaces 41 have inlet angle 42 and outlet angle 43. Inlet angle 42 is equal to 25 degrees from the tangent of turbine inlet diameter 39. Outlet angle 43 is equal to 60 degrees from the tangent of the diameter defined by turbine surface 40. Turbine control blade surfaces 41 have a curvature radius 44 equal to 0.70 inches. Motor 16 is sufficient to spin turbine blade 27 at an angular velocity up to 20,000 rpm, requiring an input power of approximately 50 watts. In the first embodiment, the pressure parametric at 30 cm H₂O stagnation pressure (max pressure, zero flow) is about 31,000,000 in² rad²/sec². The leak compensation parametric at the same angular velocity is about 46,000 in³ rad/sec

A second device was manufactured in accordance with teachings of the present invention. The second and preferred embodiment included a turbine inlet diameter 39 measures approximately 0.920-inch. The overall radius of turbine blade 27 is 1.5 inches making the diameter of turbine blade outlet surface 40 equal to 3.0 inches. Turbine control blade surfaces 41 have inlet angle 42 and outlet angle 43. Inlet angle 42 is equal to 25 degrees from the tangent of turbine inlet diameter 39. Outlet angle 43 is equal to 60 degrees from the tangent of the diameter defined by turbine outlet surface 40. Turbine control blade surfaces 41 have a curvature radius 44 equal to 0.70 inches. Motor 16 is sufficient to spin turbine blade 27 at a speed up to 18,000 rpm, requiring an input power of approximately 50 watts. In the shown preferred embodiment, the pressure parametric at 30 cm H₂O stagnation pressure (max pressure, zero flow) is about 32,000,000 in² rad²/sec². The leak compensation parametric in the second and preferred embodiment is 51,000 in³ rad/sec

Although the description above contains many details, these should not be construed as limiting the scope of the invention but as merely providing illustrations of some of the presently preferred embodiments of this invention. Therefore, it will be appreciated that the scope of the present invention fully encompasses other embodiments, which may become obvious to those skilled in the art. In the appended claims, reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the disclosure and present claims. Moreover, it is not necessary for a device or method to address every problem sought to be solved by the present invention, for it to be encompassed by the disclosure and present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

What is claimed is:

1. Ventilator device comprising:
a. an electrically powered centrifugal compressor having access to ambient air;
b. a respiratory patient support conduit having the capability to vent to the environment and in fluid communication with said centrifugal compressor;
c. a time control circuit having an “ON” and “OFF” cycle, wherein each cycle is separated by a defined time interval and being in electrical communication with said centrifugal compressor;
d. a power source in electrical communication with said time control circuit;
whereupon said time control circuit is cycled to an “ON” cycle allowing for said centrifugal compressor to supply a flow of air at a high pressure at a set voltage to said respiratory patient support conduit for a defined time interval;
whereupon expiration of said defined time interval, said time control circuit is then transitioned to an “OFF” cycle allowing for said centrifugal compressor to supply a flow of air at a low pressure at a reduced voltage to said respiratory patient support conduit and allowing said patient support conduit to vent to the environment; and
wherein at each state said centrifugal compressor simultaneously supplies compressed ambient air at a constant pressure and a variable flow rate.

2. A ventilator device as in claim 1, wherein said pressurized air has a flow rate of equal to or less than 100 liters/minute.

3. A ventilator device as in claim 1, wherein said flow of air has a high pressure of equal to or greater than 20 cm-water.

4. A ventilator device as in claim 1, wherein said flow of air has a low pressure equal to or less than 5 cm-water.

5. A ventilator device as in claim 1, wherein said centrifugal compressor comprises a turbine.

6. A ventilator device as in claim 6, wherein said turbine has a target pressure parametric within the range of 15,000,000 in² rad²/sec² and 40,000,000 in² rad²/sec².

7. A ventilator device as in claim 6, wherein said turbine has a target leak compensation parametric within the range of 30,000 in³ rad/sec and 60,000 in³ rad/sec.

8. A ventilator device as in claim 6, wherein said turbine has a stagnation pressure of equal to or greater than 30 cm-water.

9. A ventilator device as in claim 6, wherein said turbine exhibits stagnation at high pressure at a pressure parametric of greater than 30,000,000 in² rad²/sec².

10. A ventilator device as in claim 6, wherein said turbine exhibits stagnation at high pressure at a leak compensation parametric of greater than 40,000 in³ rad/sec.

11. Method for performing respiratory patient support comprising:
a. providing a ventilator device having a respiratory patient support conduit, a centrifigual compressor, and a time control circuit;
b. utilizing a centrifugal compressor capable of simultaneously supplying compressed ambient air at a constant pressure and a variable flow rate when transitioned between “ON” and “OFF” cycles;
c. connecting said respiratory patient support conduit of said ventilator device to said a patient in need of respiratory support;
d. initiating said ventilator device to have a defined time interval between “ON” and “OFF” cycles;
e. allowing said ventilator device to operate cyclicaly for as long as respiratory support is required.

12. A method for providing respiratory patient support as in claim 11, wherein said pressurized air has a flow rate of equal to or less than 100 liters/minute.

13. A method for providing respiratory patient support as in claim 11, wherein said flow of air has a high pressure of equal to or greater than 20 cm-water.

14. A method for providing respiratory patient support as in claim 11, wherein said flow of air has a low pressure equal to or less than 5 cm-water.

15. A method for providing respiratory patient support as in claim 11, wherein said centrifugal compressor comprises a turbine.

16. A method for providing respiratory patient support as in claim 15, wherein said turbine has a target pressure parametric within the range of 15,000,000 in² rad²/sec² and 40,000,000 in² rad² /sec².

17. A method for providing respiratory patient support as in claim 15, wherein said turbine has a target leak compensation parametric within the range of 30,000 in³ rad/sec and 60,000 in³ rad/sec.

18. A method for providing respiratory patient support as in claim 15, wherein said turbine has a stagnation pressure of equal to or greater than 30 cm-water.

19. A method for providing respiratory patient support as in claim 15, wherein said turbine exhibits stagnation at high pressure at a pressure parametric of greater than 30,000,000 in² rad²/sec².

20. A method for providing respiratory patient support as in claim 15, wherein said turbine exhibits stagnation at high pressure at a leak compensation parametric of greater than 40,000 in³ rad/sec.
